(19) United States
(12) Patent Application Publication
(10) Pub. No.: US 2005/0148985 A1
(43) Pub. Date: Jul. 7, 2005

(54) Absorbent article having improved waist feature

(76) Inventors: Susan K. Bronk, Greenville, WI (US); Meara C. Kelley, Menasha, WI (US); Chad M. Lang, Evanston, IL (US); Gay Lynn Wolf, Neenah, WI (US)

Correspondence Address: KIMBERLY-CLARK WORLDWIDE, INC., 401 NORTH LAKE STREET, NEENAH, WI, 54956, US

(21) Appl. No.: 10/749,908

(22) Filed: Dec. 30, 2003

(51) Int. Cl. A61F 13/15; A61F 13/20

(52) U.S. Cl. 604/387

(57) ABSTRACT

An absorbent article for absorbing body fluids and exudates, such as urine and fecal material. More particularly, an absorbent article, such as disposable diapers and adult incontinence garments, which includes an improved waist feature for improved fit and performance.

### BACKGROUND OF THE INVENTION

The present invention relates to an absorbent article for absorbing body fluids and exudates, such as urine and fecal material. More particularly, the present invention relates to absorbent garments, such as disposable diapers and adult incontinence garments, which include an improved waist feature for improved fit and performance.

Articles, particularly disposable absorbent articles may advantageously include portions that are elastic. These portions of elastic material may improve the fit, comfort and containment characteristics of the absorbent articles in use. In certain situations, these portions may be provided by separately attached elastic members that are applied to the chassis of the absorbent article at some point in the manufacturing process. For example, portions of an absorbent article that may be provided by a separately attached elastic member include the waist elastics and ear portions of the article.

However, conventional absorbent articles which incorporate a separately attached elastic member and ear portions may not be completely satisfactory for several reasons. First, when it is desired to apply a number of components to an article in a converting process, it is often necessary to include that number of separate application processes and apparatus. Each application process includes variability, when several application are utilized, the variability may be magnified resulting in undesirable articles. Second, if the separately attached components are placed on the interior of the article, the seams created may cause irritation to the user's skin. The raised portions of the seams may press into or rub the skin causing irritation. Third, every application and attachment process may present a possibility of a failure, from either a misplaced application, or a failed attachment. These failures may result in a reduction in aesthetics, performance, or may render the product unusable.

Further, although the elasticized fastening systems used on conventional absorbent articles have improved the fit of the article about the wearer, such systems have typically not been or connected to the elasticized waistbands and flaps. As a result, the fit and containment of conventional absorbent articles have not been completely satisfactory.

### SUMMARY OF THE INVENTION

The present invention is directed to providing an absorbent article including a unitary waist elastic element. Additionally, the present invention is directed to providing lateral extensions to the chassis (ear portions) which provide improved durability and integrity.

In one aspect the present invention concerns a disposable absorbent article defining two lateral end edges, two longitudinal side edges, a front waist region, a back waist region and a crotch region interconnecting the front waist region and back waist region. The disposable absorbent article includes an absorbent core defining a garment surface and a body surface; an outer cover positioned adjacent the garment surface of the absorbent core wherein the absorbent core and the outer cover define a chassis; and a unitary waist elastic element. The unitary waist elastic element defines a waist elastic element inboard portion and a pair of waist elastic element outboard portions. The unitary waist elastic element is attached to the back waist region, and the waist elastic element outboard portions extend laterally beyond the chassis of the absorbent article to provide a pair of ears. Additionally, the waist elastic element inboard portion attached to the back waist region shirrs the back waist region.

In another aspect, the present invention concerns a disposable absorbent article defining two lateral end edges, two longitudinal side edges, a front waist region, a back waist region and a crotch region interconnecting the front waist region and back waist region. The disposable absorbent article includes an absorbent core defining a garment surface and a body surface; an outer cover positioned adjacent the garment surface of the absorbent core wherein the absorbent core and the outer cover define a chassis; and a unitary waist elastic element. The unitary waist elastic element defines a waist elastic element inboard portion and a pair of waist elastic element outboard portions. The unitary waist elastic element is attached to the interior surface of the article in the back waist region along a waist elastic element upper edge in the inboard portion and along the two longitudinal side edges of the article. The waist elastic element outboard portions extend laterally beyond the chassis of the absorbent article to provide a pair of ears. The waist elastic element outboard portions are stretchable. The waist elastic element defines a waist elastic element lower edge and the waist elastic element lower edge in the inboard portion is curvilinear. Additionally, the waist elastic element inboard portion attached to the waist region shirrs the waist region.

In yet another aspect, the present invention concerns a disposable absorbent article defining two lateral end edges, two longitudinal side edges, a front waist region, a back waist region and a crotch region interconnecting the front waist region and back waist region. The disposable absorbent article includes an absorbent core defining a garment surface and a body surface; an outer cover positioned adjacent the garment surface of the absorbent core wherein the absorbent core and the outer cover define a chassis; and a unitary waist elastic element. The unitary waist elastic element defines a waist elastic element inboard portion and a pair of waist elastic element outboard portions. The unitary waist elastic element is attached to the front waist region, and the waist elastic element outboard portions extend laterally beyond the chassis of the absorbent article to provide a pair of ears. The waist elastic element inboard portion attached to the front waist region shirrs the front waist region.

Accordingly, the present invention provides a disposable absorbent article with a unitary waist elastic. As such, the unitary waist elastic may replace multiple components in the article, thereby reducing the number of seams on the interior of the absorbent article, which can in turn potentially reduce irritation to the user's skin. Moreover, a disposable absorbent article including a unitary waist elastic may be produces with fewer processes than conventional diapers having separate ear and waist elastic members thereby producing an absorbent article with less variability than conventional diapers. Still further, the present invention provides an absorbent article that may provide improved durability and integrity.

It is understood that both the foregoing general description and the following detailed description are exemplary and are intended to provide further explanation of the invention claimed. The accompanying drawings, which are incorporated in and constitute part of this specification, are included to illustrate and provide a further understanding of the article of the present invention. Together with the description, the drawings serve to explain the various aspects of the invention.

### BRIEF DESCRIPTION OF THE DRAWINGS

The present invention will be more fully understood and further advantages will become apparent when reference is made to the following detailed description of the invention and the accompanying drawings wherein like numerals represent like elements. The drawings are merely representative and are not intended to limit the scope of the appended claims.

FIG. 1 representatively illustrates a perspective view of an example of a disposable absorbent article (an infant diaper) of the present invention;

FIG. 2 representatively illustrates a plan view of the disposable absorbent article of FIG. 1 in an unfastened, stretched and laid flat condition with the surface of the article which contacts the wearers skin (interior surface) facing the viewer and with portions of the article partially cut away to show the underlying features;

FIGS. 3-5 representatively illustrate plan views of waist regions of other examples of disposable absorbent articles of the present invention, each in an unfastened, stretched and laid flat condition with the surface of the article which contacts the wearer's skin (interior surface) facing the viewer;

FIG. 4 representatively illustrates a plan view of a waist region of another example of a disposable absorbent article of the present invention in an unfastened, stretched and laid flat condition with the surface of the article which contacts the wearer's skin facing (interior surface) the viewer;

FIG. 5 representatively illustrates a plan view of a waist region of another example of a disposable absorbent article of the present invention in an unfastened, stretched and laid flat condition with the surface of the article which contacts the wearers skin (interior surface) facing the viewer; and

FIG. 6 representatively illustrates a plan view of a waist region of another example of a disposable absorbent article of the present invention in an unfastened, relaxed and laid flat condition with the surface of the article which contacts the wearer's skin facing away from the viewer (exterior surface visible).

### DETAILED DESCRIPTION OF THE INVENTION

This detailed description of the present invention will include a description of a representative absorbent article including the various components of such an article. The description of the representative absorbent article will also include a description of the features encompassed by the present invention.

The present disclosure of the invention will be expressed in terms of its various components, elements, constructions, configurations, arrangements and other features that may also be individually or collectively be referenced by the term, “aspect(s)” of the invention, or other similar terms. It is contemplated that the various forms of the disclosed invention may incorporate one or more of its various features and aspects, and that such features and aspects may be employed in any desired, operative combination thereof.

It should also be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

The present invention is directed to solving problems related to absorbent articles including providing a unitary waist elastic element. Additionally, the present invention is directed to providing lateral extension to the chassis (ear portions) which provide improved durability and integrity. In particular, the present invention in directed to an absorbent article with a unitary waist elastic element. The unitary waist elastic element may be attached by extending the portion of the unitary waist elastic element corresponding to the width of the absorbent article prior to attachment, the extended unitary waist elastic element may then be applied to the absorbent article, and then the unitary waist elastic element may be allowed to retract, shirring the waist portion of the absorbent article, as shown in FIG. 6. Alternatively, the unitary waist elastic element may be applied to the absorbent article, and then the unitary waist elastic element may be activated with heat or other means known in the art, causing the waist elastic to contract. In another alternative, the unitary waist elastic element may provide elasticized ear portion. In yet anther alternative, the unitary waist elastic element may be shaped and positioned to provide specific desired benefits.

For ease of explanation, the description hereafter will be in terms of an elastic member for use in a disposable absorbent article, specifically, a disposable diaper. Typically, disposable articles are intended for limited use and are not intended to be laundered or otherwise cleaned for reuse. For example, disposable absorbent articles that the present invention is equally adaptable for use with include training pants, feminine care products, incontinence garments, changing pads, surgical drapes, adult incontinent products, feminine care products, personal hygiene products, other personal care or health care garments, and the like.

As used herein, the term “extensible” refers to that property of a material where upon removal of an extending force, it provides a substantially permanent deformation and/or does not exhibit a significant retractive force.

As used herein, the term “elastic,” or “elastomeric” refers to that property of a material where upon removal of an extending force, it is capable of substantially recovering its original size and shape and/or exhibits a significant retractive force.

As used herein, the term “stretch,” or “stretchable” refers to a material that is either elastic or extensible. That is, the material is capable of being extended, deformed, or the like, without breaking, and may or may not significantly retract after removal of an extending force.

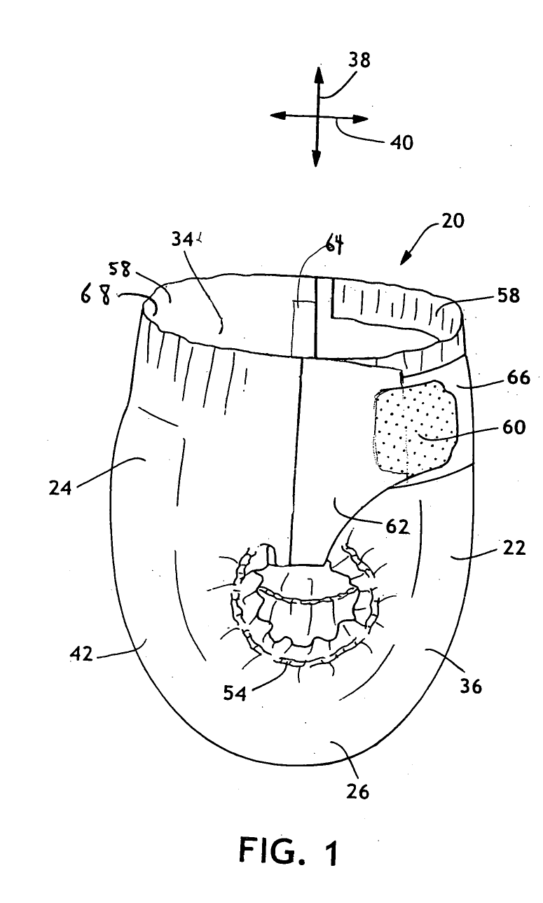

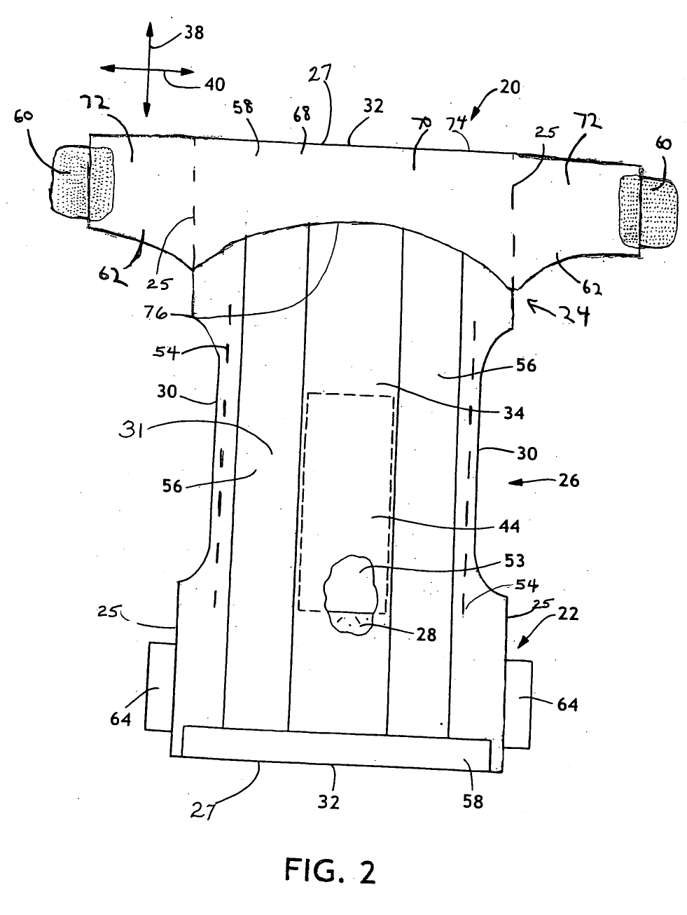

FIG. 1 representatively illustrates an example of a refastenable disposable diaper, as generally indicated at 20, of the present invention. FIG. 2 representatively illustrates the refastenable diaper of FIG. 1 in an unfastened, stretched and laid flat configuration with the surface of the diaper adapted to contact the wearer's skin facing the viewer and with portions of the diaper partially cut away to show the underlying features. As illustrated in FIG. 2, the diaper 20 defines a front waist region 22, a back waist region 24, a crotch region 26 that extends between and connects the front and back waist regions 22 and 24, a longitudinal direction 38 and a lateral direction 40. The front waist region 22 includes the portion of the diaper 20 that, when worn, is positioned on the front of the wearer while the back waist region 24 includes the portion of the diaper 20 that, when worn, is positioned on the back of the wearer. The crotch region 26 of the diaper 20 includes the portion of the diaper 20 that, when worn, is positioned between the legs of the wearer and covers the lower torso of the wearer.

The diaper 20 defines a pair of laterally opposed side edges 30, a pair of longitudinally opposed waist edges 32, an interior surface 34 that is configured to contact the wearer, and an exterior surface 36 opposite the interior surface 34 that is configured to contact the wearer's clothing in use. The illustrated diaper 20 also includes a substantially liquid impermeable outer cover 42 and a liquid permeable bodyside liner 44 that can be connected to the outer cover 42 in a superposed relation. An absorbent core 28 is located between the outer cover 42 and the bodyside liner 44. A diaper chassis 31 may be formed by the outer cover 42, the liquid permeable bodyside liner 44, and the absorbent core 28. The chassis 31 has a pair of laterally opposed side edges 25 and a pair of longitudinally opposed end edges 27. The laterally opposed side edges 30 of the diaper 20 are generally defined by the side edges of the outer cover 42 that further define leg openings that may be curvilinear. The waist edges 32 of the diaper 20 are generally defined by the waist edges of the outer cover 42 and define a waist opening that is configured to encircle the waist of the wearer when worn. The absorbent core 28 is configured to contain and/or absorb body exudates discharged from the wearer. The diaper 20 may further include leg elastics 54 and containment flaps 56 as are known to those skilled in the art. It should be recognized that individual components of the diaper may be optional depending upon the intended use of the diaper 20.

The diaper 20 may further include refastenable mechanical fasteners 60. The mechanical fasteners 60 releasably engage the opposed side edges 30 of the diaper 20 in the opposite waist regions. The mechanical fasteners 60 can include a variety of materials and surfaces known for mechanical engagement such as buttons, pins, snaps, adhesive tape fasteners, cohesives, mushroom-and-loop fasteners and hook and loop fasteners. Further, the disposable diaper 20 may include an attachment panel 66 located on the front or back waist region 22 and 24, opposite the fasteners 60 to which the fasteners 60 can be releasably engaged during use of the diaper 20. Alternatively, a variety of fastening systems may also be used to permanently or refastenably secure the front and back waist regions together.

The diaper 20 may be of various suitable shapes. For example, in the unfastened configurations as illustrated in FIG. 2, the diaper 20 may have an overall rectangular shape, T-shape or an approximately hourglass shape. In the shown embodiments, the diaper 20 has a generally I-shape in an unfastened configuration.

The various components of the diaper 20 are integrally assembled together employing various types of suitable attachment means, such as adhesive, sonic and thermal bonds or combinations thereof. In the shown embodiments, for example, the outer cover 42 and bodyside liner 44 are assembled to each other and to the absorbent core 28 with adhesive, such as a hot melt, pressure-sensitive adhesive. The adhesive may be applied as a uniform continuous layer of adhesive, a patterned layer of adhesive, a sprayed pattern of adhesive, or an array of separate lines, swirls or dots of adhesive. Alternatively, the absorbent core 28 may be connected to the outer cover 42 using conventional fasteners such as buttons, hook and loop type fasteners, adhesive tape fasteners, and the like. The other components of the diaper 20 may be suitably connected together using similar means. Similarly, other diaper components, such as the elastic members 54 and 58 and the mechanical fasteners 60, may be assembled into the diaper 20 article by employing the above-identified attachment mechanisms. Desirably, the majority of the diaper components are assembled together using ultrasonic bonding techniques for reduced manufacturing cost.

The outer cover 42 of the diaper 20, as representatively illustrated in FIG. 1, may suitably be composed of a material which is either liquid permeable or liquid impermeable. It is generally preferred that the outer cover 42 be formed from a material that is substantially impermeable to liquids. A typical outer cover 42 can be manufactured from a thin plastic film or other flexible liquid-impermeable material. For example, the outer cover 42 may be formed from a polyethylene film having a thickness of from about 0.013 millimeter (0.5 mil) to about 0.051 millimeter (2.0 mils). The materials of the outer cover 42 can be thermally or adhesively laminated together. Suitable laminate adhesives, which can be applied continuously or intermittently as beads, a spray, parallel swirls, or the like, can be obtained from Bostik-Findley, Inc., of Wauwatosa, Wis., U.S.A., or from National Starch and Chemical Company, Bridgewater, N.J., U.S.A. If it is desired to present the outer cover 42 with a more clothlike feeling, the outer cover 42 may be formed from a polyolefin film having a nonwoven web laminated to the exterior surface thereof, such as a spunbond web of polyolefin fibers. For example, a stretch-thinned polypropylene film having a thickness of about 0.015 millimeter (0.6 mil) may be thermally laminated thereto a spunbond web of polypropylene fibers. The polypropylene fibers may have a fiber diameter of about 15 to 20 microns, which nonwoven web has a basis weight of about 17 grams per square meter (0.5 ounce per square yard). The outer cover 42 may include bicomponent fibers such as polyethylene/polypropylene bicomponent fibers. Methods of forming such clothlike outer covers are known to those skilled in the art. The outer cover 42 may also be an extensible outer cover such as the outer covers described in U.S. Pat. No. 6,552,245 issued on Apr. 22, 2003 to Roessler et al. The outer cover 42 may also be a biaxially stretchable outer cover such as the outer covers described in U.S. patent application Ser. No. 09/698,517 filed on Oct. 27, 2000 by Vukos et al.

The outer cover 42 may be formed of a woven or nonwoven fibrous web layer which has been totally or partially constructed or treated to impart a desired level of liquid impermeability to selected regions that are adjacent or proximate the absorbent core 28.

Still further, the outer cover 42 may optionally be composed of a micro-porous “breathable” material which permits vapors to escape from the absorbent core 28 while still preventing liquid exudates from passing through the outer cover 42. For example, the outer cover 42 may include a vapor permeable non-woven facing layer laminated to a micro-porous film. Suitable “breathable” outer cover materials are described in U.S. Pat. No. 5,695,868 issued Dec. 9, 1997 to McCormack et al. and U.S. Pat. No. 5,843,056 issued Dec. 1, 1998 to Good et al., the descriptions of which are hereby incorporated by reference. Still further, the outer cover 42 may also be an elastomeric material such as a stretch-thermal laminate (STL), neck-bonded laminate (NBL), or stretch-bonded laminate (SBL) material. Methods of making such materials are well known to those skilled in the art and are described in U.S. Pat. No. 4,663,220 issued May 5, 1987 to Wisneski et al., U.S. Pat. No. 5,226,992 issued Jul. 13, 1993 to Morman, and European Patent Application No. EP 0 217 032 published on Apr. 8, 1987 in the names of Taylor et al., the disclosures of which are hereby incorporated by reference. The outer cover 42 can also be embossed or otherwise provided with a matte finish to provide a more aesthetically pleasing appearance.

In order to reduce the perception that the outer cover 42 feels damp or clammy, the diapers/diaper pants 20 of the invention may include a spacer or ventilation layer (not shown in Figures) between the garment-facing surface of the absorbent core 28 and the outer cover 42. The ventilation layer may include one or more nonwoven materials, for example a spunbond-meltblown-spunbond nonwoven material.

The representative absorbent articles of the invention include a bodyside liner 44 in superimposed relation to the outer cover 42. The bodyside liner 44, as representatively illustrated in FIG. 2, suitably presents a bodyfacing surface that is compliant, soft feeling, and nonirritating to the wearer's skin. Further, the bodyside liner 44 may be less hydrophilic than the absorbent core 28, to present a relatively dry surface to the wearer, and may be sufficiently porous to be liquid permeable, permitting liquid to readily penetrate through its thickness. A suitable bodyside liner 44 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers. The bodyside liner 44 is suitably employed to help isolate the wearer's skin from liquids held in the absorbent 28. The bodyside liner 44 can also be made from extensible materials as are described in U.S. Pat. No. 6,552,245 issued on Apr. 22, 2003 to Roessler et al. The bodyside liner 44 can also be made from biaxially stretchable materials as are described in U.S. patent application Ser. No. 09/698,517 filed on Oct. 27, 2000 by Vukos et al. Various woven and nonwoven fabrics can be used for the bodyside liner 44. For example, the bodyside liner may be composed of a meltblown or spunbond web of polyolefin fibers.

The bodyside liner 44 may also be a bonded-carded web composed of natural and/or synthetic fibers. The bodyside liner 44 may be composed of a substantially hydrophobic material, and the hydrophobic material may optionally be treated with a surfactant or otherwise processed to impart a desired level of wettability and hydrophilicity. In a particular embodiment of the present invention, the bodyside liner 44 is made from a nonwoven, spunbond, polypropylene fabric composed of fibers having a fiber diameter of about 21 to 23 microns formed into a web having a basis weight of about 20 grams per square meter and a density of about 0.13 grams per cubic centimeter. The fabric may be surface treated with about 0.3 weight percent of a surfactant, such as a surfactant commercially available from Hodgson Textile Chemicals, Inc. under the trade designation AHCOVEL Base N-62. The surfactant may be applied by any conventional means, such as spraying, printing, brush coating or similar techniques. The surfactant may be applied to the entire bodyside liner 44 or may be selectively applied to particular sections of the bodyside liner 44, such as the medial section along the longitudinal centerline of the diaper, to provide greater wettability of such sections. The bodyside liner 44 may further include a lotion or treatment applied thereto that is configured to be transferred to the wearer's skin. Suitable compositions for application to the bodyside liner 44 are described in U.S. Pat. No. 6,149,934 that issued to Krzysik et al. on Nov. 21, 2000.

The representative absorbent articles of the invention can include an absorbent core 28 disposed between the outer cover 42 and the bodyside liner 44. The absorbent core 28 of the diaper 20, as representatively illustrated in FIG. 1, may suitably include a matrix of hydrophilic fibers, such as a web of cellulosic fluff, mixed with particles of a high-absorbency material commonly known as superabsorbent material. In a particular aspect, the absorbent core 28 includes a matrix of cellulosic fluff, such as wood pulp fluff, and superabsorbent hydrogel-forming particles. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be nonuniformly mixed. Alternatively, the absorbent core 28 may include a laminate of fibrous webs and superabsorbent material or other suitable matrix for maintaining a superabsorbent material in a localized area.

The absorbent core 28 may have any of a number of shapes. For example, the absorbent core 28 may be rectangular, I-shaped, or T-shaped. It is generally preferred that the absorbent core 28 is narrower in the intermediate section than in the front or rear waist sections of the diaper 20. The absorbent core 28 may be provided by a single layer or, in the alternative, may be provided by multiple layers, all of which need not extend the entire length and width of the absorbent core 28. In a particular aspect of the invention, the absorbent core 28 can be generally T-shaped with the laterally extending cross-bar of the “T” generally corresponding to the front waist region 22 of the absorbent article for improved performance, especially for male infants.

The size and the absorbent capacity of absorbent core 28 should be compatible with the size of the intended wearer and the liquid loading imparted by the intended use of the absorbent article. Further, the size and the absorbent capacity of the absorbent core 28 can be varied to accommodate wearers ranging from infants through adults. In addition, it has been found that with the present invention, the densities and/or basis weights of the absorbent core 28 can be varied.

The high-absorbency material may be selected from natural, synthetic, and modified natural polymers and materials. The high-absorbency materials may be inorganic materials, such as silica gels, or organic compounds, such as crosslinked polymers. The term “crosslinked” refers to methods for effectively rendering normally water-soluble materials substantially water insoluble but swellable. Such methods include, for example, physical entanglement, crystalline domains, covalent bonds, ionic complexes and associations, hydrophilic associations such as hydrogen bonding, and hydrophobic associations or Van der Waals forces.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinyl morpholinone), poly(vinyl alcohol), and mixtures and copolymers thereof. Further polymers suitable for use in the absorbent core 28 include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum, and the like. Mixtures of natural and wholly or partially synthetic absorbent polymers can also be useful in the present invention.

The high absorbency material may be in any of a wide variety of geometric forms. As a general rule, it is preferred that the high absorbency material be in the form of discrete particles. However, the high absorbency material may also be in the form of fibers, flakes, rods, spheres, needles, or the like. In general, the high absorbency material is present in the absorbent core 28 in an amount of from about 5 to about 90 percent by weight, desirably in an amount of at least about 30 percent by weight, and even more desirably in an amount of at least about 50 percent by weight based on a total weight of the absorbent core 28. For example, in a particular aspect, the absorbent core 28 may include a laminate which includes at least about 50 percent by weight and desirably at least about 70 percent by weight of high-absorbency material overwrapped by a fibrous web or other suitable material for maintaining the high-absorbency material in a localized area.

An example of high-absorbency material suitable for use in the present invention is DRYTECH 2035 polymer available from Dow Chemical, a business having offices in Midland, Mich. Other suitable superabsorbents may include FAVOR SXM 880 polymer obtained from Stockhausen, a business having offices in Greensboro, N.C.

Optionally, a substantially hydrophilic tissue or nonwoven wrapsheet (not illustrated) may be employed to help maintain the integrity of the structure of the absorbent core 28. The wrapsheet is typically placed about the absorbent core 28 over at least the two major facing surfaces thereof. The wrapsheet may be composed of an absorbent cellulosic material, such as creped wadding or a high wet-strength tissue. In one aspect of the invention, the wrapsheet may be configured to provide a wicking layer that helps to rapidly distribute liquid over the mass of absorbent fibers constituting the absorbent core 28.

Due to the thinness of absorbent core 28 and the high absorbency material within the absorbent core 28, the liquid uptake rates of the absorbent core 28, by itself, may be too low, or may not be adequately sustained over multiple insults of liquid into the absorbent core 28. To improve the overall liquid uptake and air exchange, the diaper 20 may further include a porous, liquid-permeable layer of surge management material 53, as representatively illustrated in FIG. 2. The surge management layer 53 is typically less hydrophilic than the absorbent core 28, and has an operable level of density and basis weight to quickly collect and temporarily hold liquid surges, to transport the liquid from its initial entrance point and to substantially completely release the liquid to other parts of the absorbent core 28. This configuration can help prevent the liquid from pooling and collecting on the portion of the diaper 20 positioned against the wearer's skin, thereby reducing the feeling of wetness by the wearer. The structure of the surge management layer 53 also generally enhances the air exchange within the diaper 20.

Various woven and nonwoven fabrics can be used to construct the surge management layer 53. For example, the surge management layer 53 may be a layer composed of a meltblown or spunbond web of synthetic fibers, such as polyolefin fibers. The surge management layer 53 may also be a bonded-carded-web or an airlaid web composed of natural and synthetic fibers. The bonded-carded-web may, for example, be a thermally bonded web that is bonded using low melt binder fibers, powder or adhesive. The webs can optionally include a mixture of different fibers. The surge management layer 53 may be composed of a substantially hydrophobic material, and the hydrophobic material may optionally be treated with a surfactant or otherwise processed to impart a desired level of wettability and hydrophilicity. In a particular aspect, the surge management layer 53 includes a hydrophobic, nonwoven material having a basis weight of from about 30 to about 120 grams per square meter.

The absorbent articles of the invention can include additional components. For example, as representatively illustrated in FIGS. 1 and 2, the disposable diaper 20 may include a pair of containment flaps 56 that are configured to provide a barrier to the lateral flow of body exudates. The containment flaps 56 may be located along the laterally opposed side edges 30 of the diaper adjacent the side edges of the absorbent core 28. Each containment flap 56 typically defines an unattached edge that is configured to maintain an upright, perpendicular configuration in at least the crotch region 26 of the diaper 20 to form a seal against the wearer's body. The containment flaps 56 may extend longitudinally along the entire length of the absorbent core 28 or may only extend partially along the length of the absorbent core 28. When the containment flaps 56 are shorter in length than the absorbent core 28, the containment flaps 56 can be selectively positioned anywhere along the side edges 30 of diaper 20 in the crotch region 26. In a particular aspect of the invention, the containment flaps 56 extend along the entire length of the absorbent core 28 to better contain the body exudates. Such containment flaps 56 are generally well known to those skilled in the art.

The diaper 20 of the different configurations of the present invention may further include elastics at the waist edges 32 and side edges 30 of the diaper 20 to further prevent leakage of body exudates and support the absorbent core 28. For example, as representatively illustrated in FIGS. 1 and 2, the diaper 20 of the present invention may include a pair of leg elastic members 54 that are connected to the laterally opposed side edges 30 of the diaper 20 in the crotch region 26. As will be discussed in greater detail below, the diaper 20 may also include a pair of waist elastic members 58 that is connected to the longitudinally opposed waist edges 32 of the diaper 20. The leg elastics 54 and waist elastics 58 are generally adapted to fit about the legs and waist of a wearer in use to maintain a positive, contacting relationship with the wearer to effectively reduce or eliminate the leakage of body exudates from the diaper 20.

Materials suitable for use as the leg elastics 54 are well known to those skilled in the art. Exemplary of such materials are sheets or strands or ribbons of a polymeric, elastomeric material that may be adhered to the outer cover 42 in a stretched position, or that may be attached to the outer cover 42 while the outer cover is pleated, such that elastic constrictive forces are imparted to the outer cover 42. The leg elastics 54 may also include such materials as polyurethane, synthetic and natural rubber. The leg elastics 54 may be formed by attaching separate pieces of stretchable material to the leg regions of the article. For example, the leg elastics 54 may include a piece of stretch-bonded laminate material attached to the interior surface 34 of the article. Elasticity may be added or incorporated into the leg opening of absorbent articles utilizing a variety of know approaches.

The absorbent articles of the invention may include one or more components that extend laterally outward from the laterally opposed side edges 25 of the chassis 31. Components that extend laterally outward may include front ear portions 64 and back ear portions 62. The front ear portions 64 and the back ear portions 62 may be formed of one or more materials and may include laminates of materials. The front ear portions 64 and the back ear portions 62 may improve the fit of the absorbent article. More specifically, the front ear portions 64 may provide additional coverage around the waist of the wearer and they may assist caregivers with positioning the front waist region 22 on the wearer of the article. The back ear portions 62 may also provide coverage around the waist of the wearer. More specifically, the back ear portions 62 may provide the bridging material between the back waist region 24 of the article and the front waist region 22 such that the back ear portions 62 form part of the article's waist opening and an upper edge of the article's leg openings. Additionally, the back ear portions 62 may include fastening materials that facilitate joining of the back waist region 24 with the front waist region 22. For example, the back ear portions 62 may include fasteners 60 selected for engagement with an attachment panel 66 in the front waist region 22 of the article.

Presently available infant diapers typically include back ear portions 62 that include a stretchable material. When the back ear portions 62 include a stretchable material, the back ear portions 62 may increase the range over which the fasteners 60 may be engaged into the attachment panel 66 or directly into the outer cover 42. Further, when the back ear portions 62 include a stretchable material, the article may be worn by a greater range of users as a result of the increased fit range. An exemplary material from which the back ear portions 62 may be constructed is a necked bonded laminate material having two nonwoven (e.g. spunbond) facings with an elastomeric film (e.g. KRATON film) laminated in between. Other suitable stretchable materials are known in the art. Depending on the design of the article, it may also be desirable for the front ear portions 64 to include a stretchable material.

The back ear portions 62 and the waist elastic 58 in the back waist region 24 or the front ear portion 64 and the front waist region 22 may be provided by a unitary waist elastic element 68. The unitary waist elastic element 68 may include a unitary waist elastic element inboard portion 70 located generally within the laterally opposed side edges 25 of the chassis 31. The unitary waist elastic element 68 may also include a unitary waist elastic element outboard portion 72 located generally outside the laterally opposed side edges 25 of the chassis 31. The unitary waist elastic element 68 may include a unitary waist elastic element upper edge 74 which is the edge of the unitary waist elastic element 68 which is the edge closest to the longitudinally opposed waist edge 32. The unitary waist elastic element 68 may include a unitary waist elastic element lower edge 76 which is the edge closest to the crotch region 26. Additionally, both the unitary waist elastic element upper edge 74 and the unitary waist elastic element lower edge 76 may be divided into portions that correspond to the unitary waist elastic element inboard portion 70 and outboard portion 72.

The unitary waist elastic element 68 may be composed of a substantially elastomeric material, such as a stretch-bonded-laminate (SBL) material, a neck-bonded-laminate (NBL) material, an elastomeric film, an elastomeric foam material, or the like, which is elastomerically stretchable at least along the lateral direction 40. For example, suitable meltblown elastomeric fibrous webs for forming the unitary waist element 68 are described in U.S. Pat. No. 4,663,220 issued May 5, 1987 to T. Wisneski et al., the entire disclosure of which is hereby incorporated by reference. Examples of composite fabrics comprising at least one layer of nonwoven textile fabric secured to a fibrous elastic layer are described in European Patent Application EP 0 217 032 A2 published on Apr. 8, 1987 which has the listed inventors of J. Taylor et al., the entire disclosure of which is hereby incorporated by reference. Examples of NBL materials are described in U.S. Pat. No. 5,226,992 issued Jul. 13, 1993 to Mormon, the entire disclosure of which is hereby incorporated by reference.

The elastomeric material from which the unitary waist elastic element 68 is formed may suitably be capable of being elongated by at least about 50 percent, alternatively by at least about 100 percent, alternatively by at least about 130 percent. After elongation to 50 percent (if the elastomeric material is capable of being elongated to no more than 100 percent) or 100 percent (if the elastomeric material is capable of being elongated to more than 100 percent), the elastomeric material suitably recovers to at least about 50 percent of its original length, alternatively to at least about 80 percent of its original length. The elastomeric material may be an inherently elastomeric material, that is, one which is formed in an elastomeric state, or may be rendered elastomeric through processing subsequent formation. For example, the elastomeric material may be heat or pressure activated.

When the unitary waist elastic element 68 includes a stretchable material, the back ear portions 62 may increase the range with which the fasteners 60 may be engaged into the attachment panel 66 or directly into the outer cover 42. Further, when the unitary waist elastic element 68 includes a stretchable material, the article may be worn by a greater range of users as a result of the increased fit range.

The unitary waist elastic element 68 may be provided by 1) stretching an elastic element, 2) attaching the stretched elastic element to a waist region of an absorbent article, 3) releasing the stretched elastic element, allowing the stretched elastic element to retract thereby providing a shirred, gathered waist region (as shown in FIG. 6). This process of providing this unitary waist elastic element may be performed by the method and apparatus as disclosed in the concurrently filed patent application Ser. No. ______ entitled METHOD AND APPARATUS FOR APPLYING AN ELASTIC MEMBER by Hoffman et al. which was contemporaneously filed Dec. 30, 2003 (attorney docket No. 18,996), the entire disclosure of which is incorporated herein by reference in a manner that is consistent (not contradictory) herewith. Alternatively, the unitary waist elastic element 68 may be provided by 1) providing a latent elastic element, 2) attaching the latent elastic element to a waist region of an absorbent article, 3) activating the latent elastic element, causing the latent elastic element to retract thereby providing a shirred, gathered waist region (as shown in FIG. 6).

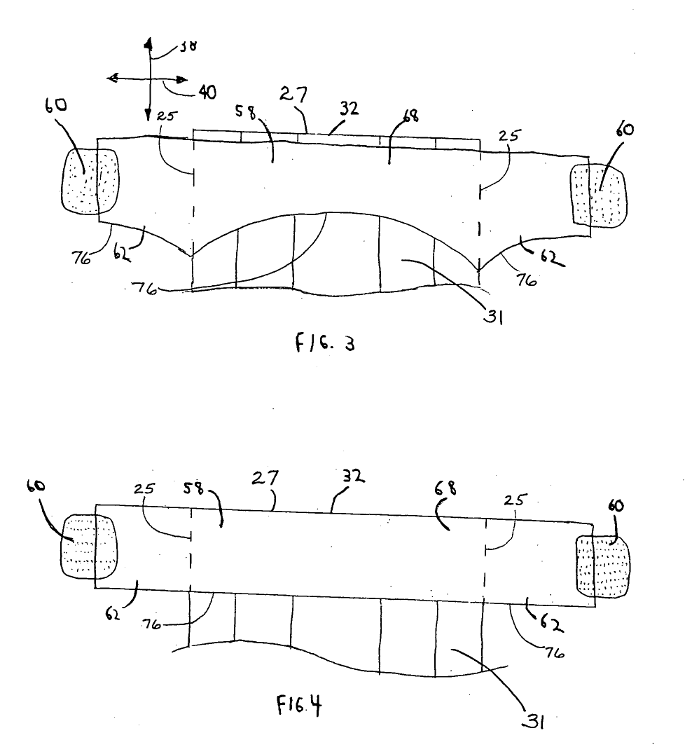

The unitary waist elastic element 68 may be shaped and positioned in many ways to provide the benefits desired in a particular application (as shown in FIGS. 2-5). The unitary waist elastic element 68 may be cut in the same process that cuts the longitudinally opposed waist edges 32. In this case the unitary waist elastic element upper edge 74 and a longitudinally opposed waist edge will be coterminous. As shown in FIG. 2. Alternatively if the unitary waist elastic element 68 is cut and positioned on the absorbent article separate from the process that cuts the longitudinally opposed waist edges 32, variability in the both the positioning and the cutting process may require distance between the unitary waist elastic element upper edge 74 and the longitudinally opposed waist edge 32 be at least 3 mm, alternatively at least 4 mm, alternatively at least 5 mm, alternatively at least 6 mm, alternatively at least 7 mm, alternatively at least 8 mm, alternatively at least 9 mm, and finally alternatively 10 mm, as shown in FIG. 3.

The unitary waist elastic element lower edge 76 may be of numerous shapes. The lower edge 76 may be linear as shown in FIG. 4 to minimize manufacturing complexities. Alternatively, the unitary waist elastic element lower edge 76 may contain a plurality of curves. In particular, the unitary waist elastic element lower edge 76 in the inboard portion may be curvilinear as shown in FIG. 3. More specifically, the unitary waist elastic element lower edge 76 in the inboard portion may be the shape of an arc of a circle. More specifically, the inventors have discovered that when the unitary waist elastic element 68 is attached to the interior surface of the article, and when the radius of curvature of the unitary waist elastic element lower edge 76 in the inboard portion is less than 25 cm and is not attached to the absorbent article inboard of the laterally opposed side edges 25 of the chassis 31, an application of force, as is seen in use, may cause the unitary waist elastic element lower edge 76 to pull away from the interior surface of the chassis 31 forming a pocket, which may reduce or prevent body exudates from escaping past the unitary waist elastic element 68 and thereby soiling the user's clothing.

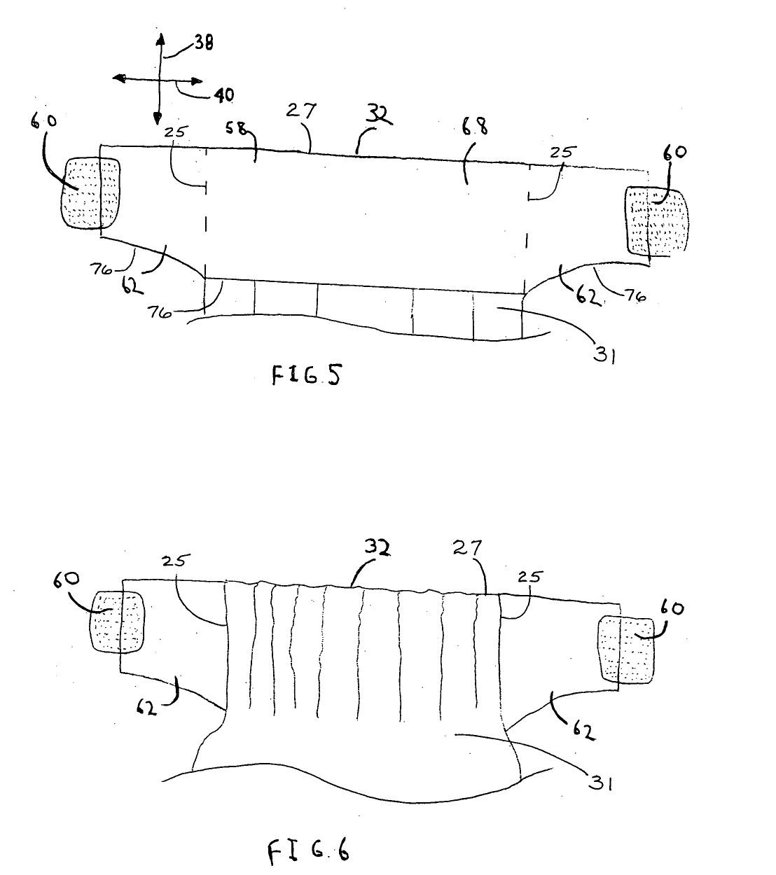

The unitary waist elastic element lower edge 76 in the outboard portion may be shaped to provide benefits independent from the unitary waist elastic element lower edge 76 in the inboard portion. The unitary waist elastic element outboard portion 72 may form all or part of the front or back ear portions 62, 64. The unitary waist elastic element lower edge 76 in the outboard portion may be shaped to provide a curved leg cut-out, as shown in FIG. 5.

These curved leg cut-outs may improve aesthetics and contribute to the functionality of the element as the relative size of the waist of the user becomes larger in comparison to the absorbent article. As the waist of the user becomes larger the unitary waist elastic element lower edge 76 in the outboard portion forms more of the circumference of the leg cut-out. The unitary waist elastic element lower edge 76 in the outboard portion may be the shape of an arc of a circle to provide a more aesthetically pleasing look as well as functionally providing more uniform tension around the leg which may improve fit. More specifically, when the radius of curvature of the unitary waist elastic element lower edge 76 in the outboard portion is less than 25 cm, the visual transition between the opposed side edges 30 which form part of the leg opening, and the unitary waist elastic element lower edge 76 in the outboard portions which also forms part of the leg opening, becomes smoother and more aesthetically pleasing. In addition, when the radius of curvature of the unitary waist elastic element lower edge 76 in the outboard portion is less than about 25 cm, the forces that the unitary waist elastic element 68 apply to the opposed side edges may become more uniform, which may reduce stress concentration on the chassis 31 of the diaper, and which may also reduce pressure concentrations applied to the user, thereby improving comfort.

These features of the outboard portion and the inboard portion of the unitary waist elastic lower edge 76 may be combined. For example, as shown in FIG. 3, the unitary waist elastic element lower edge 76 in the outboard portion has a concave shape, in addition the unitary waist elastic element lower edge 76 in the inboard portion also has a concave shape. In this example the unitary waist elastic element lower edge 76 has a “w” shape.

The unitary waist elastic element 68 may be attached to the chassis 31 employing various types of suitable attachment means, such as adhesive, sonic and thermal bonds or combinations thereof. For example, the unitary waist elastic element 68 and the chassis 31 can be bonded to each other with adhesive, such as a hot melt, pressure-sensitive adhesive. The adhesive may be applied as a uniform continuous layer of adhesive, a patterned layer of adhesive, a sprayed pattern of adhesive, or an array of separate lines, swirls or dots of adhesive. One pattern that may be used to attach the unitary waist elastic element 68 to the chassis 31 may include a lateral line of adhesive positioned near the unitary waist elastic element upper edge 74, as well as two longitudinal lines of adhesive positioned near the opposed side edges 30, the adhesive forming three sides of a rectangle. In addition to these three sides of a rectangle of adhesive, additional adhesive may attach the unitary waist elastic element lower edge 76 in the inboard portion to the chassis 31, along either a portion or along the entire length of the unitary waist elastic element lower edge 76.

The unitary waist elastic element 68 may be attached to the chassis 31 in several locations; for example, the unitary waist elastic element 68 may be attached between the outer cover 42 and the bodyside liner 44. Alternatively, the unitary waist elastic element 68 may be attached to the outside of the outer cover 42. And alternatively still, the unitary waist elastic element 68 may be attached to the interior of the absorbent article, more specifically to the inside of the bodyside liner 44. The unitary waist elastic element 68 may be attached to the front waist region 22 or the back waist region 24 or both the front waist region 22 and the back waist region 24.

Having described the invention in rather full detail, it will be readily apparent that various changes and modifications can be made without departing from the spirit of the invention. All of such changes and modifications are contemplated as being within the scope of the invention as defined by the subjoined claims.

1. A disposable absorbent article defining two lateral end edges, two longitudinal side edges, a front waist region, a back waist region and a crotch region interconnecting the front waist region and back waist region, the disposable absorbent article comprising:
an absorbent core defining a garment surface and a body surface;
an outer cover positioned adjacent the garment surface of the absorbent core;
wherein the absorbent core and the outer cover define a chassis; and
a unitary waist elastic element defining a waist elastic element inboard portion and a pair of unitary waist elastic element outboard portions, wherein the unitary waist elastic element is attached to the back waist region, and wherein the unitary waist elastic element outboard portions extend laterally beyond the chassis of the absorbent article to provide a pair of ears, and wherein the unitary waist elastic element inboard portion shirrs the back waist region.

2. The disposable absorbent article of claim 1 wherein the unitary waist elastic element is attached to an interior surface of the article.

3. The disposable absorbent article of claim 1 wherein the unitary waist elastic element further defines a unitary waist elastic element upper edge, and the unitary waist elastic element upper edge of the unitary waist elastic inboard portion is coterminous with a lateral end edge of the disposable absorbent article.

4. The disposable absorbent article of claim 1 wherein the unitary waist elastic element further defines a unitary waist elastic element upper edge, and the unitary waist elastic element upper edge of the unitary waist elastic inboard portion is at least 5 mm from a lateral end edge of the disposable absorbent article.

5. The disposable absorbent article of claim 1 wherein the unitary waist elastic element defines a unitary waist elastic element lower edge and the unitary waist elastic element lower edge of the inboard portion is curvilinear.

6. The disposable absorbent article of claim 5 wherein the unitary waist elastic element lower edge of the inboard portion has a radius of curvature of less than 25 cm.

7. The disposable absorbent article of claim 1 wherein the unitary waist elastic element further defines a unitary waist elastic lower edge and the unitary waist elastic element lower edge defines a “w” shape.

8. The disposable absorbent article of claim 1 wherein the unitary waist elastic element further defines a unitary waist elastic element upper edge and the unitary waist elastic element upper edge of the inboard portion is attached to an interior surface of the article.

9. The disposable absorbent article of claim 8 wherein the unitary waist elastic element further defines a unitary waist elastic element lower edge wherein at least a portion of the unitary waist elastic element lower edge of the inboard portion is attached to the interior surface of the article.

10. The disposable absorbent article of claim 1 wherein the unitary waist elastic element further defines a unitary waist elastic element lower edge wherein the unitary waist elastic element lower edge of the outboard portions are configured to provide curved leg cut-outs.

11. The disposable absorbent article of claim 10 wherein the curved leg cut-outs have a radius of curvature of less than 25 cm.

12. The disposable absorbent article of claim 1 further comprising a fastener attached to each of the unitary waist elastic element outboard portions.

13. The disposable absorbent article of claim 1 wherein the waist region containing the unitary waist elastic element is elastic.

14. The disposable absorbent article of claim 1 further comprising a second unitary waist elastic element defining a second unitary waist elastic element inboard portion and a second pair of unitary waist elastic element outboard portions, wherein the second unitary waist elastic element is attached to the front waist region, and wherein the second unitary waist elastic element outboard portions extend laterally beyond the chassis of the absorbent article to provide a second pair of ears, and wherein the second unitary waist elastic element inboard portion attached to the front waist region shirrs the front waist region.

15. A disposable absorbent article defining two lateral end edges, two longitudinal side edges, a front waist region, a back waist region and a crotch region interconnecting the front waist region and back waist region, the disposable absorbent article comprising:
an absorbent core defining a garment surface and a body surface;
an outer cover positioned adjacent the garment surface of the absorbent core, wherein the absorbent core and the outer cover define a chassis; and
a unitary waist elastic element defining a unitary waist elastic element inboard portion and a pair of unitary waist elastic element outboard portions, wherein the unitary waist elastic element is attached to the interior surface of the article to the back waist region along a unitary waist elastic element upper edge in the inboard portion, wherein the unitary waist elastic element outboard portions extend laterally beyond the chassis of the absorbent article to provide a pair of ears, wherein the unitary waist elastic element outboard portions are stretchable, wherein the unitary waist elastic element defines a unitary waist elastic element lower edge and the unitary waist elastic element lower edge in the inboard portion is curvilinear; and wherein the unitary waist elastic element inboard portion attached to the back waist region shirrs the back waist region.

16. The disposable absorbent article of claim 15 wherein the unitary waist elastic element upper edge of the inboard portion is adjacent a lateral end edge of the disposable absorbent article.

17. The disposable absorbent article of claim 15 wherein the unitary waist elastic element upper edge of the inboard portion is at least 5 mm from a lateral end edge of the disposable absorbent article.

18. The disposable absorbent article of claim 15 further comprising a fastener attached to each of the outboard portions of the waist elastic element.

19. A disposable absorbent article defining two lateral end edges, two longitudinal side edges, an interior surface, an exterior surface, a front waist region, a back waist region and a crotch region interconnecting the front waist region and back waist region, the disposable absorbent article comprising:
an absorbent core defining a garment surface and a body surface;
an outer cover positioned adjacent the garment surface of the absorbent core;
wherein the absorbent core and the outer cover define a chassis; and
a unitary waist elastic element defining a waist elastic element inboard portion and a pair of waist elastic element outboard portions, wherein the unitary waist elastic element is attached to the front waist region, and wherein the waist elastic element outboard portions extend laterally beyond the chassis of the absorbent article to provide a pair of ears, and wherein the waist elastic element inboard portion attached to the front waist region shirrs the front waist region.

20. The disposable absorbent article of claim 19 wherein the waist elastic element upper edge in the inboard portion is adjacent a lateral end edge of the disposable absorbent article.

21. The disposable absorbent article of claim 19 wherein the waist elastic element upper edge in the inboard portion is at least 5 mm from a lateral end edge of the disposable absorbent article.

22. The disposable absorbent article of claim 19 further comprising a fastener attached to each of the outboard portions of the waist elastic element.
